(19) United States
(12) Patent Application Publication
(10) Pub. No.: US 2013/0196323 A1
(43) Pub. Date: Aug. 1, 2013

(54) Methods Of Nucleic Acid Analysis

(71) Applicant: The University of North Carolina At Greensboro, Greensboro, NC (US)

(72) Inventors: Adam Hall, Burlington, NC (US); Vincent C. Henrich, Greensboro, NC (US)

(73) Assignee: The University of North Carolina At Greensboro, Greensboro, NC (US)

(21) Appl. No.: 13/757,420

(22) Filed: Feb. 1, 2013

Related U.S. Application Data
(60) Provisional application No. 61/593,695, filed on Feb. 1, 2012.

(51) Int. Cl. C12Q 1/68; G01N 33/487

(52) U.S. Cl. CPC C12Q 1/689; G01N 33/48721; Y10S 977/781; Y10S 977/92; B82Y 15/00
USPC 435/6.11; 436/501; 977/781; 977/920

(57) ABSTRACT

In one aspect, methods of nucleic acid analysis are described herein. In some embodiments, a method of nucleic acid analysis comprises providing a mixture of differing single-strand nucleic acid segments, including unamplified single-strand nucleic acid segments, combining the mixture of differing single-strand nucleic acid segments with a single-strand nucleic acid probe, contacting the mixture with a membrane comprising at least one nanopore, applying an electric field across the nanopore, and measuring change in current through the nanopore during one or more nucleic acid translocation events.

### CROSS REFERENCE TO RELATED APPLICATION

This application claims priority pursuant to 35 U.S.C, §119(e) to U.S. Provisional Patent Application Ser. No. 61/593,695, filed on Feb. 1, 2012, which is hereby incorporated by reference in its entirety.

### FIELD

The present invention relates to methods of molecular biological analysis and, in particular, to nucleic acid analysis.

### SEQUENCE LISTING

The sequence listing in the text file seqlist.txt created on Jan. 30, 2013 and having a size of 10,000 bytes is incorporated herein by reference.

### BACKGROUND

The analysis of mixtures of nucleic acids such as ribonucleic acid (RNA) and deoxyribonucleic acid (DNA) has become increasingly important in a variety of applications, including medical diagnostics and genotyping. Therefore, a number of tools have been developed in recent years to perform nucleic acid analysis.

Some methods of analysis rely on amplification of one or more nucleic acids through a polymerase chain reaction (PCR) or other enrichment step. In addition, some methods rely on purification of one or more analyte nucleic acids. Further, some methods also require the use of fluorescent probes or other labeling techniques. However, such sample preparation or preprocessing steps can introduce systematic biases into measurements, increase the cost and time needed to analyze a nucleic acid mixture and destroy pertinent information characterizing the environmental background from which the nucleic acid mixture was prepared.

### SUMMARY

Briefly, in one aspect, methods of nucleic acid analysis are described herein which, in some embodiments, may provide one or more advantages over prior methods. For example, a method described herein can be used to analyze unamplified and/or unpurified nucleic acids at low detection limits, such as about 0.1 femtomolar (fM). Moreover, in some embodiments, a method described herein can be used to quantify nucleic acid content of a sample without loss of data characterizing the environmental background from which the sample was collected. For example, methods described herein can be employed to analyze the presence nucleic acid(s) of target species present in water samples, including pathogenic species, in efforts to monitor water quality and/or identify origins of disease.

A method of nucleic acid analysis described herein, in some embodiments, comprises providing a mixture of differing single-strand nucleic acid segments including unamplified single-strand nucleic acid segments, combining the mixture of differing single-strand nucleic acid segments with a single-strand nucleic acid probe, contacting the mixture with a membrane comprising at least one nanopore, applying an electric field across the nanopore and measuring change in current through the nanopore during one or more nucleic acid translocation events. In some embodiments, a translocation event comprises passage of an unamplified target single-strand nucleic acid segment hybridized with the single-strand nucleic acid probe through the nanopore. Translocation events can also comprise passage through the nanopore of self-hybridized single-strand nucleic acid demonstrating secondary structure. Moreover, a method described herein further comprises quantifying the unamplified target single-strand nucleic acid segments of the mixture. In some embodiments, the membrane comprises an array of nanopores.

These and other embodiments are described in greater detail in the detailed description which follows.

### BRIEF DESCRIPTION OF THE FIGURES

FIG. 1 illustrates an exploded perspective view of an apparatus suitable for carrying out a method according to one embodiment described herein.

FIG. 2 illustrates a cross sectional view of the apparatus of FIG. 1 taken along line A-A′.

### DETAILED DESCRIPTION

Embodiments described herein can be understood more readily by reference to the following detailed description and drawings. Elements, apparati, and methods described herein, however, are not limited to the specific embodiments presented in the detailed description and drawings. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

In one aspect, methods of nucleic acid analysis are described herein. In some embodiments, a method of nucleic acid analysis comprises providing a mixture of differing single-stranded nucleic acid segments including unamplified single-strand nucleic acid segments, combining the mixture of differing single-strand nucleic acid segments with a single-strand nucleic acid probe, contacting the mixture with a membrane comprising at least one nanopore, applying an electric field across the nanopore, and measuring change in current through the nanopore during one or more nucleic acid translocation events. In some embodiments, the membrane comprises a plurality of nanopores across which an electric field is applied, and changes in current through the nanopores are measured during nucleic acid translocation events. As described further herein, a translocation event comprises passage of an unamplified target single-strand nucleic acid segment hybridized with the single-strand nucleic acid probe. Translocation events can also comprise passage of self-hybridized single-strand nucleic acid demonstrating secondary structure.

Moreover, methods described herein further comprise detecting and quantifying target single-strand nucleic acid segments in the mixture, including unamplified target single-strand nucleic acid segments. In some embodiments, single-strand nucleic acid segments of the mixture differing from target segments can also be detected and quantified. For example, a differing single-strand nucleic acid segment present in the mixture is not hybridized with a single-strand nucleic acid probe but instead self-hybridizes to provide one or more secondary structure geometries. Such a single-strand nucleic acid segment can be detected instead of or in addition to the target single-strand nucleic acid segment present in the mixture, as described further hereinbelow.

Turning now to specific steps, methods of nucleic acid analysis described herein comprise providing a mixture of differing single-strand nucleic acid segments. Differing single-strand nucleic acid segments, in some embodiments, comprise nucleic acid segments having differing numbers and/or sequences of nucleotides. As descried herein, differing nucleic acid segments can arise from a genomic or metagenomic DNA sample subjected to factors such as digestion by restriction or other modifying enzymes, shearing or sonication, followed by exposure to heat and/or other chemicals affecting DNA helix stability.

A nucleic acid segment can have any length not inconsistent with the objectives of the present invention. For example, in some embodiments, a single-strand nucleic acid segment comprises about 15 to about 500 bases. In some embodiments, a single-strand nucleic acid segment is about 50 to 500 bases. In addition, a mixture of differing single-strand nucleic acid segments can comprise any type of nucleic acid. In some embodiments, for example, single-strand nucleic acid segments of a mixture comprise ribonucleic acid (RNA) and/or deoxyribonucleic acid (DNA). Further, the RNA and/or DNA can comprise any type of RNA and/or DNA not inconsistent with the objectives of the present invention. In some embodiments, for instance, RNA comprises mRNA, rRNA, tRNA, siRNA, miRNA or combinations or mixtures thereof. DNA, in some embodiments, comprises A-DNA, B-DNA, Z-DNA, rDNA, cDNA or combinations or mixtures thereof. Additionally, in some embodiments, a mixture of differing single-strand nucleic acid segments includes unamplified single-strand nucleic acid segments. Thus, in some embodiments, a method described herein can be used to quantify the unamplified concentration of a single-strand nucleic acid segment in a sample or mixture.

Further, a mixture of differing single-strand nucleic acid segments can be in an unpurified state. In some embodiments, for instance, an unpurified mixture is derived from a water sample including one or more species having intact genomic, metagenomic or mitochondrial DNA, such as one or more pathogens. Metagenomic DNA can contain sequences from more than one genome and generally describes an environmental DNA sample or any sample that contains more than one species and often includes an entire community of organisms or microorganisms. A pathogen, in some embodiments, comprises a bacterium or fungus. A mixture of single-strand nucleic acid segments can ultimately be obtained from such pathogenic genomic or mitochondrial DNA through use of one or more restriction enzymes. Suitable restriction enzymes are selected according to the nucleic acid segment(s) of interest and generally recognize cleavage sites of 4 to 8 nucleotides in double strand DNA. One non-limiting example of a restriction enzyme suitable for use in some embodiments described herein is Sau3A1 (commercially available from New England Biolabs, Inc.). In some embodiments, several restriction enzymes are used separately or together. Cleaved nucleic acid segments in double-stranded form can undergo strand separation by heating to provide single-strand nucleic acid segments for analysis according to methods described herein.

In being in an unpurified state, the mixture can further comprise various chemical species in addition to the single-strand nucleic acid segments. In some embodiments, for example, large nucleic acid segments having greater than 1000, 5000, 10,000 or 1 million base pairs can be present in the mixture as by-products of restriction enzyme processes. Such by-products can be present in significantly higher concentrations than target single-strand nucleic acid segments or other single-strand nucleic segments of interest. In some embodiments, for example, a mixture comprises one or more large nucleic acid segments and/or other large molecules at a concentration 2-5 orders of magnitude greater than the molar concentration of a target single-strand nucleic acid segment in the mixture.

The use of unpurified mixtures comprising unamplified target single-stranded nucleic acids is in sharp contrast to prior methods. In several prior methods, the presence of large nucleic acid segments or other large molecules is avoided as such species can inhibit the efficient operation of the method and/or prevent the achievement of a desired analyte detection limit. Further, large nucleic acid segments have the potential to occlude nanopore structure and/or precipitate protracted translocation events precluding or inhibiting complete analysis of the nucleic acid mixture.

Alternatively, a mixture of differing single-strand nucleic acid segments is purified. Purification can be carried out in any manner not inconsistent with the objectives of the present invention. For example, in some embodiments, purification can be carried out by providing one or more solid state probes to the mixture. A solid state probe, in some embodiments, comprises a bead, such as a magnetic bead, functionalized with a probe specific to one or more target single-strand nucleic acid segments of the mixture. The solid state probe, for example, can comprise a single-strand nucleic acid segment complementary to the target single-strand nucleic acid segment of the mixture. Thus, in some embodiments, the bead can bond target single-strand nucleic acid segments. The mixture comprising single-strand nucleic acid unhybridized with the solid state probe is subsequently removed from the solid state probe. In some embodiments, binding and/or other kinetic models for solid state probes and target single-strand nucleic acids can be used to determine uptake levels of the target single-strand nucleic acids by the solid state probes. Such models can assist in determining target single-strand nucleic acid concentration in the sample in conjunction with nanopore analytical methods described herein.

Solid state probe comprising bound or hybridized target single-strand nucleic acid is introduced into a fresh or new medium for analysis of the target-single strand nucleic acid. Hybridized target single-strand nucleic acid can be released or eluted from the beads or solid state substrate by various methods including chemical displacement of target single-strand nucleic acid hybridized with complementary probe single-strand nucleic acid. Elution conditions can be chosen such that all or substantially all nucleic acid is released into the new media. Analysis of the target single-strand nucleic acid is then administered according to a method described herein.

Single-strand nucleic acid probes are used to hybridize with target single strand nucleic acid segments for subsequent detection and quantification by the nanopore membrane. In some embodiments, a single-strand nucleic acid probe comprises a chemical species capable of sequence-specific binding to a single-strand nucleic acid segment targeted in the mixture. In some embodiments, for instance, a single-strand nucleic acid probe comprises a protein. In some embodiments, a single-strand nucleic acid probe comprises a nucleic acid segment that is complementary to one or more single-strand nucleic acid segments targeted in the mixture. Further, a single-strand nucleic acid probe comprising a nucleic acid can comprise any type of nucleic acid not inconsistent with the objectives of the present invention. In some embodiments, for example, a single-strand nucleic acid probe comprises RNA or DNA. Alternatively, in other embodiments, a single-strand nucleic acid probe comprises locked nucleic acid (LNA). Non-limiting examples of single-strand nucleic acid probes for the identification of pathogenic species in a sample, such as a water sample according to methods described herein, include single-strand nucleic acid segments provided in Table 1.

TABLE I
Single-strand nucleic acid probes of pathogenic species

| | GenBank | | SEQ ID |
|---|---|---|---|
| Template* | Accession No. | Probe Sequence (5′-3′) | NO: |
| Escherichia | | AGCTAATACCGCATAACGTCGCAAGACCAA | 1 |
| coli | | AGAGGGGGACCTTCGGGCCT | |
| Escherichia | | AAGACCAAAGAGGGGGACCTTCGGGCCTCT | 2 |
| coli | | TGCCATCGGATGTGCCCAGA | |
| Yersinia | | AAGAGCAAAGTGGGGGACCTTAGGGCCTCA | 3 |
| pestis | | CGCCATCGGATGAACCCAGA | |
| Yersinia | AF366383 | TCTGGGTTCATCCGATGGCGTGAGGCCCTAA | 4 |
| pestis | | GGTCCCCCACTTTGCTCTT | |
| 16S OTU | | TCACCAGTTTTACCCTAGGCGGCTCCTTACG | 5 |
| 77 + 80.8 | | GTTACCGACTTTAGGTACACCCGGCTTCC | |
| 16S OTU | | TCGGCCACACCGTGGCAAGCGCCCCCCTTGC | 6 |
| 80.8_20 | | GGTTAAGCTACCTGCTTCTGGTGCAACAA | |
| 16S OTU | | CTAGTTACCAGTTTTACCCTAGGCAGCTCCT | 7 |
| 80.8_26 | | TGCGGTCACCGACTTCAGGCACCCCCAGC | |
| 16S OTU | | GAACCCTGCCGTGGTAATCGCCCTCCTTGCG | 8 |
| 80.8_2 | | GTTAGGCTAACTACTTCTGGCAGAACCCG | |
| 16S OTU | | ACCAGCCTTACCTTAGGACGCTGCCCCCTTG | 9 |
| 80.8_4a | | CGGTTGGCGTGCATACTTCGGGTGCGACC | |
| 16S OTU | | ACCAGCCTTACCTTAGGACGCTGCCCCCTTG | 10 |
| 80.8_4b | | CGGTTGGCGCGCATACTTCGGGTGCGACC | |
| 18S OTU | | CAACTCTCGCGGGGAGGGATGTATTTATTAG | 11 |
| 80.8_2 | | ATAAAAAACCAATGCGGGTTCTGCTCGCC | |
| 18S OTU | | ACTTTACGAAGGGGCGCTTTTATTAGATCAA | 12 |
| 80.8_20 | | AATCAATCAGGAGCAATCCTGTTTTTGTG | |
| 18S OTU | | GACCCGACGCAAGGACGGTCGCATTTATTA | 13 |
| 77_54 | | GAACAAAGCCATCCGGTCCCCGGGACCGTA | |
| 18S OTU | | TGCGGGACGAGCGCATTTATTAGAACAAAA | 14 |
| 80.8_34 | | CCATCCGGACTCTCGCGAGTCCGTTGCTGG | |
| 18S OTU | | CATTTTGGGAAACTATGGCTAATACATGCTT | 15 |
| 77_6 | | ACAGACCTTCGGGTTGTATTTATTAGTTT | |
| 18S OTU | | GACCTTCGGAAAGAGCGCATTTATTAGACCA | 16 |
| 77_73 | | AAACCAGTCGAGTTTCGGCTTGTTTGTTG | |
| 18S OTU | | CAATACCCTTCTGGGGTAGTATTTATTAGAA | 17 |
| GL_59 | | AGAAACCAACCCCTTCGGGGTGATGTGGT | |
| 18S OTU | | ACGAACGAGCGCATTTATTAGAGCAAAACC | 18 |
| GL_8 | | AATCAGGTTTCGGCCTGTCTTTTGGTGAAT | |
| 18S OTU | | CCCCGACTTCGGAAGGGGTGTATTTATTAGA | 19 |
| 80.8_66a | | TAAAAAACCAATGCCCTTCGGGGCTACTT | |
| 18S OTU | | CCCCAACTTCGGGAGGGGTGTATTTATTAGA | 20 |
| 80.8_66b | | TAAAAAACCAACGCCCTTCGGGGCTTCTT | |
| Cryposporidium | AF222998 | GATTTCTCATAAGGTGCTGAAGGAGTAAGG | 21 |
| parvum | | AACAACCTCCAATCTCTAGT | |
| Entamoeba | X65163 | GAAATGTCTTATTGACATCCCCTCAGCATTG | 22 |
| histolytica | | TCCCATGCTTGAATATTCA | |
| Giardia | AF199449 | CCCACGCGGCGGGTCCAACGGGCCTGCCTG | 23 |
| intestinalis | | GAGCGCTCCCGTTTCCTCGT | |
| Microsporidium | AY140647 | CTTTATCATCGGACTCGCCCCTGGCCAGCGC | 24 |
| sp. STF | | TTTCGCCTCTGTCGCTCCT | |
| OTU LT1A42, | | CAAGCAGAAAGGCACGCGCGCACCGTCCAA | 25 |
| multi-copy, | | CCAGAGGCTGACAGTTCACA | |
| identified as | | | |
| Cryptomonas | | | |
| sp., strain | | | |
| M420 | | | |
| OTU LT1A4, | | GCACGCGCATGCCGTCCGACCAGAGGCCGA | 26 |
| multi-copy, | | CAGCCCACACGCGCCCAAAA | |
| identified as | | | |
| Cryptomonas | | | |
| ovata, strain | | | |
| CCAP 979/61 | | | |
| Bacillus | AB116124 | GTGACAGCCGAAGCCGCCTTTCAATTTTCGA | 27 |
| anthracis | | ACCATGCGGTTCAAAATGTT | |
| strain: S51 | | | |
| Francisella | AY243028 | AGGCTCATCCATCTGCGACACGCCGAAAGC | 28 |
| tularensis | | CACCTTTAATCCACAGATAT | |
| strain 3523 | | | |
| Legionella | AJ496383 | AATCCTTAAAAGTCGGTCGTAGTCCGGATTG | 29 |
| pneumophilia | | GAGTCTGCAACTCGACTCC | |
| serogroup 6 | | | |
| Salmonella | Z49264 | CTTGGTGAGCCGTTACCTCACCAACAAGCTA | 30 |
| typhimurium | | ATCCCATCTGGGCACATCT | |
| Vibrio | X76337 | ATCCCACCTGGGCATATCCGGTAGCGCAAG | 31 |
| cholerae | | GCCCGAAGGTCCCCTGCTTT | |
| CECT 514 T | | | |
| Uncultured | AF233412 | TATTCATAAGGTACATACAAAACACCACAC | 32 |
| human fecal | | GTGGCGAACTTTATTCCCTT | |
| bacterium | | | |
| HF74 | | | |
| Uncultured | AF233408 | TATTCATAAAGTACATGCAAACGGGTATGCA | 33 |
| human fecal | | TACCCGACTTTATTCCTTT | |
| bacterium HF8 | | | |
| Uncultured | AF233413 | TATTCATACGGTACATACAAAAAGGCACAC | 34 |
| human fecal | | GTGCCTCACTTTATTCCCGT | |
| bacterium | | | |
| HF10 | | | |
| Burkolderia | AB091761 | AGGCCCGAAGGTCCCCCGCTTTCATCCGTAG | 35 |
| cepacia | | ATCGTATGCGGTATTAATC | |

*OTU = microbial operational taxonomic unit

In some embodiments, probes can also comprise complements to the single-strand nucleic acid segments listed in Table I, the complements running in an antiparallel 5′-3′ direction. Further, single-strand nucleic acid probes can be custom synthesized according to nucleic acid synthetic procedures known to one of ordinary skill in the art. In addition, single-strand nucleic acid probes described herein can also be obtained from commercial suppliers, such as Integrated DNA Technologies or Sigma.

In some embodiments, single-strand nucleic acid segments of interest can be in double-stranded form following treatment of a mixture with one or more restriction enzymes. To facilitate hybridization with a single-strand nucleic acid probe, the mixture can be heated to induce strand separation. Strand separation can be generally achieved by heating the mixture to a temperature greater than 90° C., such as 95° C. The single-strand nucleic acid probe is added to the mixture of single-strand nucleic acid segments, and the mixture is cooled to effectuate hybridization/annealing of the probes with target single-strand nucleic acid. For example, the mixture is cooled to 65-80° C. to induce specific hybridization interactions. In some embodiments, the mixture is cooled to a temperature just below the melting point of the hybridized complex consisting of the probe and the target single-strand nucleic acid. Hybridization of target single-strand nucleic acid segments with nucleic acid probes occurs prior to translocation of the target single-strand nucleic acid segments. Further, in some embodiments, the hybridized double-strand nucleic acid segment is only one portion of a longer nucleic acid segment that also comprises one or more single-strand portions. In such embodiments, the single stranded portions that flank the hybridized double strand nucleic acid segment can be removed through treatment with 5′ to 3′ exonuclease and 3′ to 5′ exonuclease activities such as found with Mung bean nuclease (New England Biolabs, Inc.) prior to translocation.

Additionally, in some embodiments, single-strand nucleic acid segments not hybridized with a single-strand nucleic acid probe undergo self-hybridization providing one or more secondary structure geometries. Self-hybridization of non-target single-strand nucleic acid segments can occur prior to translocation of such segments. In some embodiments, single-strand nucleic acid secondary structures include random or statistical coils. In some embodiments, for example, a single-strand nucleic acid secondary structure is globular. A globular secondary structure can have a diameter smaller than the diameter of a nanopore of a membrane described herein. Alternatively, a globular secondary structure has a diameter larger than the diameter of a nanopore of a membrane described herein. In some embodiments, a secondary structure has a diameter of less than about 100 nm or less than about 50 nm. In some embodiments, a secondary structure has a diameter between about 10 nm and about 20 nm, between about 20 nm and about 50 nm, or between about 50 nm and about 100 nm.

The mixture of nucleic acids is contacted with a membrane comprising at least one nanopore. In some embodiments, the membrane comprises an array of nanopores. The membrane can have any thickness and be formed from any material not inconsistent with the objectives of the present invention. In some embodiments, a membrane is non-metallic. A non-metallic membrane can comprise one or more electrically insulating materials, including ceramic materials. Suitable ceramics include metal oxides, metal nitrides, metal carbides or metal carbonitrides or combinations thereof. In some embodiments, a ceramic suitable for use as a membrane is silicon nitride (SiN, Si₃N₄). Additionally, a membrane ceramic can comprise silicon oxide, silicon carbide, aluminum oxide or a transition metal oxide.

In some embodiments, a ceramic membrane is polycrystalline in nature. In some embodiments, a ceramic membrane is single crystalline in nature. Moreover, a ceramic membrane can be multilayered. Individual layers of a multilayered membrane can comprise the same material or divergent materials. In some embodiments, individual layers of a ceramic membrane are independently selected from the group consisting of transition metal carbide, transition metal nitride, transition metal carbonitride, transition metal oxide, alumina, silica and silicon nitride.

Further, a membrane can comprise one or more semiconducting materials. In some embodiments, suitable semiconducting materials include II/VI semiconductors, Group IV semiconductors or III/V semiconductors. In some embodiments, a semiconductor material comprises a ternary semiconductor or a quaternary semiconductor. Suitable semiconductor materials can have an amorphous structure, crystalline structure or mixture thereof. Crystalline semiconductor materials can be polycrystalline or single crystalline.

In some embodiments, a membrane is metallic. In such embodiments, a membrane can be a metal or various alloys of metals. In some embodiments, for example, suitable metals are transition metals, including noble metals such as gold. Alternatively, a membrane, in some embodiments, is not gold. Metallic membranes can be coated with dielectric or electrically insulating materials for use in methods described herein.

In some embodiments, a membrane comprises an organic material. For example, a membrane can comprise one or more polymeric materials. Suitable polymeric materials include thermoplastics, thermosets or elastomers. A polymeric material, in some embodiments, comprises one or more of polyethylene, polypropylene, and polycarbonate.

Membranes suitable for use methods described herein can have any desired thickness. In some embodiments, a membrane has a thickness suitable for detecting and/or conducting analysis of one or more nucleic acid segments, including single-strand nucleic acid segments. In some embodiments, a membrane has an average thickness less than about 200 nm or less than about 100 nm. In some embodiments, a membrane has an average thickness according to Table II.

```
TABLE II
Nanopore Membrane Thicknesses (nm)
Membrane Thickness (nm)
<50
  1-30
10-20
20-50
  50-100
100-500
250-750
```

Further, a membrane can have a thickness on the atomic scale. In some embodiments, a membrane has a thickness less than 1 nm, such as 0.1 nm to 0.9 nm. In some embodiments, the thickness of a membrane is measured prior to nanopore formation according to a method described herein.

In addition, a nanopore of a membrane described herein can have any size and shape not inconsistent with the objectives of the present invention. In some embodiments, for example, at least one nanopore has a diameter greater than about 1 nm or greater than about 5 nm. A nanopore of a membrane described herein can have a diameter according to Table III.

```
TABLE III
Nanopore Diameter (nm)
Nanopore Diameter
>10
  1-20
  1-10
1-5
  5-10
10-15
10-20
1.5-4
```

Further, a nanopore can have a thickness commensurate with the average thickness of the membrane. Therefore, in some embodiments, a nanopore can have a thickness selected from Table II herein. Alternatively, a nanopore has a thickness less than the average thickness of the membrane.

Moreover, the diameter and/or thickness of a nanopore can be selected based on a desired signal to noise ratio (SNR) of a measurement described herein, such as a current measurement associated with a translocation event. The SNR of a translocation event, in some embodiments, is higher for larger diameter nanopores and lower for smaller diameter nanopores. Additionally, in some embodiments, the diameter and/or thickness of a nanopore are selected based on a desired dwell time of a translocated species in the nanopore or a desired duration of a translocation event. In some embodiments, the dwell time and/or the duration of a translocation event is longer for a thicker nanopore than for a thinner nanopore. Dwell time, in some embodiments, is the time elapsed from an initial conductance drop in the nanopore until its return to the baseline value.

A membrane described herein can be formed in any manner not inconsistent with the objectives of the present invention. In some embodiments, for instance, a membrane is formed according to a method described in Patent Cooperation Treaty (PCT) Application Publication WO 2012/170499, the entirety of which is hereby incorporated by reference.

In some embodiments, contacting a mixture described herein with a nanopore membrane is carried out without purifying or enriching the mixture. In some embodiments, contacting a mixture with a membrane is carried out without amplifying target single-strand nucleic acid segments of the mixture.

Methods of nucleic acid analysis described herein also comprise applying an electric field across at least one nanopore and measuring change in current through the nanopore during one or more nucleic acid translocation events. An electric field can be applied across a nanopore in any manner not inconsistent with the objectives of the present invention. For example, an applied electric field can have any desired strength and/or duration. In some embodiments, an electric field is continuously applied. An applied electric field, in some embodiments, has a voltage of between about 10 mV and about 2 V. In some embodiments, the voltage is between about 50 mV and about 1 V or between about 100 mV and about 800 mV. An electric field can be applied by placing a membrane described herein between electrically isolated ionic solutions and providing voltage across the solutions with electrodes.

Measuring a change in current through a nanopore can be carried out in any manner not inconsistent with the objectives of the present invention. In some embodiments, current is measured at a rate of about 50 kHz to about 500 kHz or about 100 kHz to about 300 kHz. Moreover, in some embodiments, a change in current is not measured immediately following application of an electric field as described herein. Instead, measurement is commenced following an electric transient period in which current drift may be observed. A transient period, in some embodiments, may last for up to 3 seconds or up to 5 seconds. In some embodiments, measurement of current is commenced after a current baseline has been established that is free or substantially free of current drift.

In addition, current measurement can be administered using a low noise amplifier attached to the electrodes and/or other electrical components, such as a computer and/or a voltammeter. In some embodiments, a computer is used to record, digitize and/or analyze measured current changes. A computer, in some embodiments, comprises one or more forms of memory for data storage, at least one element that carries out arithmetic and logic operations, and a sequencing and control element that can change the order of operations based on one or more parameters, such as stored information. In some embodiments, a computer comprises a central processing unit (CPU) and a portioned memory system. A computer, in some embodiments, is operable to store and execute a computer program product. Further, in some embodiments, measured current is low pass filtered before being digitized, such as low pass filtered at 100 kHz.

In some embodiments, one or more translocation events comprise passage of a target single-strand nucleic acid segment hybridized with a single-strand nucleic acid probe described herein. The target single-strand nucleic acid segment can be unamplified and residing in an unpurified mixture containing nucleic acids and/or other molecular species not of interest. In some embodiments, one or more translocation events comprise passage of a nucleic acid species other than a probe hybridized single-strand nucleic acid segment, such as passage of a self-hybridized single-strand nucleic acid segment exhibiting secondary structure.

In some embodiments, a target single-strand nucleic acid segment comprises about 50 to about 500 bases and can be cut from genomic or mitochondrial DNA of a pathogenic species using one or more restriction enzymes. As recognized by one of skill in the art, mitochondrial DNA is only available for eukaryotic pathogens. Moreover, a target single-strand nucleic acid segment can be a non-conserved or unique sequence of genomic or mitochondrial DNA, including DNA of a pathogen such a bacterium or fungus. For example, in some embodiments, a target single-strand nucleic acid segment comprises a unique sequence of Escherichia coli (E. coli) DNA or Yersinia pestis (Y. pestis) DNA. Unique sequences, in some embodiments, can be found in generally conserved genes. In some embodiments, a target single-strand nucleic acid segment comprises sequencing complementary to a single-strand nucleic acid probe described herein, including a probe listed in Table 1 herein. Thus, methods described herein can be used to analyze a mixture (such as a water sample) for the presence of a pathogen such as a bacterium or fungus, including a pathogen listed in Table 1.

Methods described herein further comprise detecting a target single-strand nucleic acid segment present in a nucleic acid mixture. The target single-strand nucleic acid segment can be unamplified and part of a mixture of other nucleic acid species not of interest. In some embodiments, the detection limit of an unamplified target single-strand nucleic acid segment present in a mixture is less than about 10 fM. In some embodiments, the detection limit is about 1 fM.

Detecting a target single-strand nucleic acid segment present in a mixture can comprise correlating a measured current to the translocation of one or more particular species of nucleic acid segments through a nanopore. For example, in some embodiments, translocation of a particular species can be correlated to a change in the magnitude of the measured current across a nanopore. In some embodiments, a smaller change in current is correlated to the translocation of a target single-strand nucleic acid segment hybridized with a single-strand nucleic acid probe, and a larger change in current is correlated to the translocation of single-strand nucleic acid segments, including the translocation of a single-strand nucleic acid having secondary structure. It should be noted, however, that the absolute magnitude of a current change associated with a particular species can vary based on the thickness and/or diameter of the nanopore through which the species is translocated. A current change, in some embodiments, ranges between about 1 pA and about 300 pA. In some embodiments, a larger current change described herein is at least about two times a smaller current change. In some embodiments, a larger current change is between about 2 times and about 10 times or between about 2 times and about 5 times a smaller current change.

Alternatively, in some embodiments, translocation of a particular species can be correlated to the duration of a translocation event. The duration of a translocation event, is the time period over which a change in the magnitude of the measured current across a nanopore is observed, such as a time period over which a smaller change in current or a larger change in current is continuously or substantially continuously observed. Therefore, in some embodiments, the duration of a translocation event corresponds to the dwell time of a particular species in a nanopore described herein, such as the dwell time of a probe hybridized nucleic acid segment in a nanopore. In some embodiments, for instance, a shorter translocation event or dwell time is correlated to the translocation of a target single-strand nucleic acid segment hybridized with a single-strand nucleic acid probe, and a longer translocation event or dwell time is correlated to the translocation of an unhybridized or single-strand nucleic acid segment. Dwell time of a particular species, in some embodiments, can depend on the diameter and/or thickness of the nanopore through which the translocation occurs. In some embodiments, a dwell time ranges between about 1 μs and about 1000 μs. In some embodiments, for instance, a shorter dwell time is less than about 200 μs or less than about 100 μs. In some embodiments, a shorter dwell time is between about 15 μs and about 150 μs or between about 40 μs and about 60 μs. In contrast, a longer dwell time can be greater than about 100 μs or greater than about 200 μs. A longer dwell time can range between about 200 μs and about 1000 μs or between about 200 μs and about 500 μs. Therefore, in some embodiments, a longer dwell time is up to about 10 times the duration of a shorter dwell time.

Additionally, in some embodiments, detecting a target single-strand nucleic acid segment present in a mixture comprises correlating a change in magnitude of current and duration of the current change to the translocation of one or more particular species of nucleic acid segments through a nanopore. Further, in some embodiments of methods described herein, detecting one or more target single-strand nucleic acid segments by correlating a measured current change and/or duration of the current change to the translocation of one or more particular species can be carried out in real time.

In addition, a method described herein further comprises quantifying one or more translocated species, such as one or more target single-strand nucleic acid segments of the mixture. Quantifying one or more nucleic acid segments of the mixture can be carried out in any manner not inconsistent with the objectives of the present invention. In some embodiments, for example, quantifying comprises classifying a smaller change in current as the translocation of a probe hybridized target single-strand nucleic acid segment and a larger change in current as the translocation of single-strand nucleic acid segments, including the translocation of a single-strand nucleic acid having secondary structure, and counting or otherwise tabulating the occurrence/frequency of each classified translocation event during the analysis. In some embodiments, quantifying comprises comparing the number of translocations of a first nucleic acid segment to the number of translocations of a second nucleic acid segment. In some embodiments, quantifying comprises calculating the number of translocations of a first and/or second nucleic acid segment, including the number of translocations per unit volume of the combined mixture. Further, in some embodiments, quantifying one or more translocated species can be carried out in real time. In other embodiments, quantifying can be carried out following a desired number of observations of translocations, such as up to 1000 translocations, up to 5000 translocations, or up to 10,000 translocations.

In some embodiments wherein the magnitude and/or duration of current change through a nanopore is correlated with the identity of a translocating species, one or more current change and/or duration thresholds are provided to categorize the translocating species. In some embodiments, for example, a translocating species inducing a current change and/or duration less than a predetermined threshold can be classified as a single-strand nucleic acid segment hybridized with a single-strand nucleic acid probe. Similarly, in some embodiments, translocating species inducing a current change and/or duration in excess of a predetermined threshold can be classified as single-strand nucleic acid segments, including single-strand nucleic acid having secondary structure. In some embodiments, the relative number of events less than a predetermined threshold among all measured events can yield the relative amount of target single-strand nucleic acid segments in a mixture. In some embodiments, one or more current change and/or duration threshold tables are provided for the quantification and/or characterization of translocating species in a single strand nucleic acid mixture described herein. For example, some possible current change (ΔI) and duration (t) thresholds are provided in Table IV below.

TABLE IV
Thresholds.

| Shorter t (μs) | Longer t (μs) | Smaller ΔI (pA) | Larger ΔI (pA) |
|---|---|---|---|
| ≦50 | >50 | ≦20 | >20 |
| ≦100 | >100 | ≦60 | >60 |
| ≦200 | >200 | ≦150 | >150 |
| 15-150 | 200-500 | ≦200 | >200 |
| 40-60 | 200-1000 | 1-150 | 200-300 |

Methods described herein provide a rapid detection and analysis of target nucleic acids in a nucleic acid mixture without reference to providing a complete or substantially complete sequencing analysis of the target nucleic acids. As a result, desired nucleic acid and/or biomarker information of a biological system can be quickly ascertained according to one or more methods described herein without resorting to time consuming and costly sample amplification and sequencing procedures. Further, the ability to analyze a sample without amplification of target nucleic acid sequences and/or purification of the sample mixture retains valuable information characterizing the environmental background from which the nucleic acid mixture was prepared.

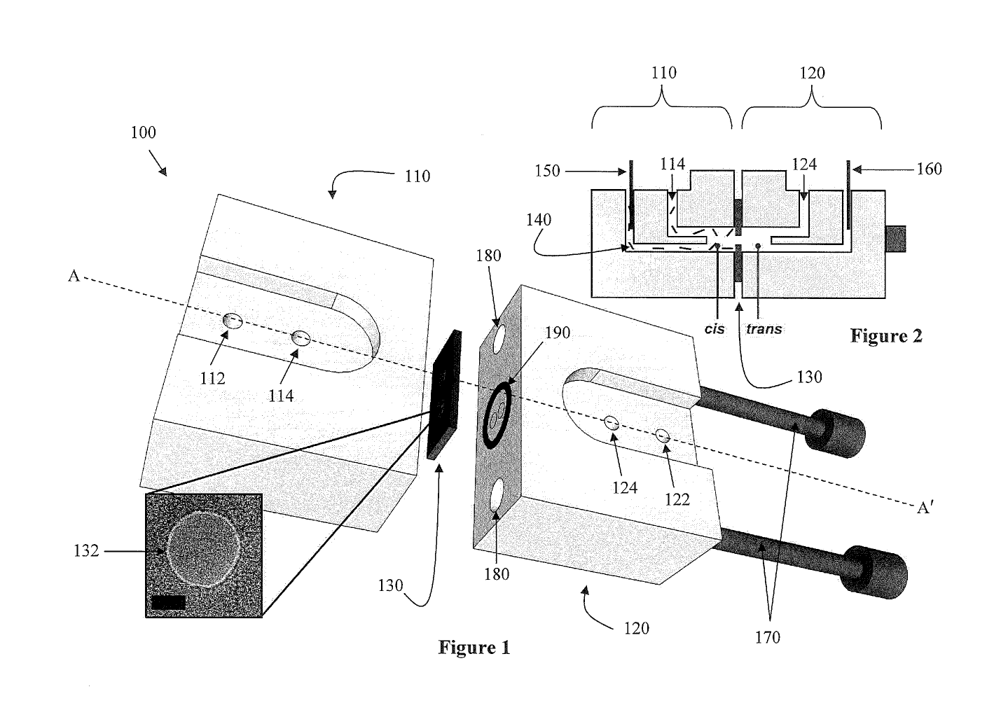

Turning now to the figures, FIG. 1 illustrates an exploded perspective view of an apparatus or flow cell suitable for carrying out a method according to one embodiment described herein. The apparatus of FIG. 1 is illustrated schematically to provide clarity, and the various elements depicted in FIG. 1 are not necessarily drawn to scale. FIG. 2 illustrates a cross sectional view of the apparatus of FIG. 1 taken along line A-A′.

The apparatus (100) in the figures comprises a first chamber (110) and a second chamber (120) separated by a membrane (130). The membrane (130) can comprise any membrane described herein. As illustrated in the figures, first chamber (110) forms a “cis” (or “inside”) side of the membrane (130) and second chamber (120) forms a “trans” (or “outside”) side of the membrane (130). However, other arrangements are also possible. In the embodiment of the figures, a mixture of differing single-strand nucleic acid segments (140) is disposed in the interior of the first chamber (110) of the apparatus (100). Any mixture described herein may be used. An electrolyte such as an aqueous ionic solution (not shown) is also disposed in the interior of both the first chamber (110) and the second chamber (120). Any electrolyte not inconsistent with the objectives of the present invention may be used. For example, in some embodiments, an electrolyte comprises one or more of KCl, LiCl, NaCl, tris-HCl, and EDTA (ethylenediaminetetraacetic acid). The electrolyte, in some embodiments, is an ionic solution of 10mM to 8M. The membrane (130) disposed between the first chamber (110) and the second chamber (120) comprises at least one nanopore (132). The nanopore can have any configuration of a nanopore described herein. In the embodiment of FIG. 1, the nanopore (132) is illustrated by a transmission electron microscope (TEM) image in an exploded view. The scale bar in the TEM image is 5 nm. As described herein, one or more single-strand nucleic acid segments (140) can translocate from the first chamber (110) of the apparatus (100) to the second chamber (120) of the apparatus (100) through the nanopore (132).

The apparatus (100) also comprises a first conduit (112) and a second conduit (114) that provide fluid communication with and access to the first chamber (110) of the apparatus (100) from outside the apparatus (100). Similarly, the apparatus (100) also comprises a third conduit (122) and a fourth conduit (124) providing fluid communication with and access to the second chamber (120) of the apparatus (100) from outside the apparatus (100). The conduits (112, 114, 122, 124) can be used to bring the mixture of single-strand nucleic acid segments (140) into contact with the membrane (130) and/or to add an electrolyte to the first (110) and/or second (120) chamber. For example, a mixture can be added to the first (110) and/or second (120) chamber through one of the conduits (112, 114, 122, 124), such as the second conduit (114).

The conduits (112, 114, 122, 124) can also be used to provide electrodes in the first (110) and/or second (120) chamber. As illustrated in FIG. 2, a first electrode (150) is disposed in the first chamber (110) of the apparatus (100) through the first conduit (112) and a second electrode (160) is disposed in the second chamber (120) of the apparatus (100) through the third conduit (122). The electrodes are omitted from FIG. 1 for the sake of clarity. First and second electrodes (150, 160) can comprise any electrodes not inconsistent with the objectives of the present invention. For example, in some embodiments, an electrode comprises an Ag/AgCl electrode. In the embodiment of the figures, the first and second electrodes (150, 160) are depicted schematically as wire electrodes electrically connected to one another through a circuit (not shown) outside of the chambers (110, 120) of the apparatus (100). In some embodiments, for example, the electrodes can be connected to a patch clamp amplifier, such as an Axon 200B amplifier (available from Axon Instruments). However, other arrangements of electrodes and additional electrical components are also possible. The first and second electrodes (150, 160) can be used to apply an electric field across the nanopore (132). The first and second electrodes (150, 160) can also be used to measure the current through the nanopore (132), including during one or more translocation events, as described herein. The apparatus (100) can also comprise additional components (not shown) for carrying out a method described herein, such as one or more voltameters and/or computers.

In the embodiment of the figures, the first (110) and the second (120) chambers of the apparatus (100) can be held together using male components (170) and female components (180). In the figures, the female components (180) are visible only in the second chamber (120) of the apparatus (100). However, female components (180) are also disposed in the first chamber (110). Any male and female components not inconsistent with the objectives of the present invention may be used. In some embodiments, for instance, the male components (170) comprise pins or screws and the female components (180) comprise channels or threads. Thus, by tightening or loosening the screws, the apparatus (100) can be held together or taken apart as needed for cleaning, repair, or refurbishment.

When the first (110) and second (120) chambers of the apparatus (100) are held together by the male (170) and female (180) components, one or more o-rings (190) can be used to ensure that a material disposed in the chambers (110, 120) and conduits (112, 114, 122, 124) can only move from the cis side of the membrane (130) to the trans side of the membrane (130) by translocating through the nanopore (132). In the figures, only one o-ring (190) is visible. However, a second o-ring (not shown) can also be present on the first chamber (110) of the apparatus (100) in an analogous fashion.

The first (110) and second (120) chambers of the apparatus (100) can be formed from any non-conductive or substantially non-conductive material not inconsistent with the objectives of the present invention. In some embodiments, for instance, a chamber is formed from a dielectric material, including an organic or inorganic material. In some embodiments, a chamber is formed from a polymer such as polyethylene, polypropylene, or polycarbonate. In some embodiments, a chamber is formed from a ceramic or inorganic oxide such as alumina or titania.

FIGS. 1 and 2 illustrate one possible apparatus that can be used to carry out a method according to one embodiment described herein. However, other apparatus can also be used. In some embodiments, for example, a membrane is disposed in a Perspex flow cell. Further, in some embodiments, an apparatus or flow cell suitable for use in a method described herein is a handheld apparatus.

In one embodiment, a method described herein can be carried out using the apparatus of the figures as follows. First, a mixture of single-strand nucleic acid segments (140) is prepared in a manner described herein and introduced into the first chamber (110) through the second conduit (114). Then, the first and second electrodes (150, 160) are used to apply an electric field across the membrane (130). A voltammeter, low noise amplifier, and computer electrically coupled to the electrodes (150, 160) are used to measure current in the nanopore (132). Once a baseline current is established, the current in the nanopore is observed for an extended period of time at a frequency described herein, such as 200 kHz. Each time the current changes from the baseline a sufficient amount to cross a translocation event threshold described herein, the computer of the apparatus records the current change and the dwell time for the translocation event. This process continues until a desired number of translocation events (such as 1000 to 2000 events) are observed and recorded. Then, the computer is used to assign each translocation event to the translocation of a particular species of the mixture, such as a target single-strand nucleic acid segment (e.g., in hybridized form with a probe described herein) or a non-target nucleic acid segment (e.g., in the form of a self-hybridized secondary structure globule), based on the measured current change and/or dwell time associated with each type of species, as described hereinabove. The absolute and relative numbers of translocation events of each type can then be used to derive various data regarding the mixture, such as the presence or absence of an analyte or target single-strand nucleic acid segment or the concentration of an analyte or target nucleic acid segment in the mixture. In addition, in some embodiments described herein, the assignment and quantification of translocation events can be carried out by the computer in real time as the measurements are being conducted, rather than after the entirety of the desired number of events have been observed.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

That which is claimed is:

1. A method of nucleic acid analysis comprising:
providing a mixture of differing single-strand nucleic acid segments, including unamplified single-strand nucleic acid segments;
combining the mixture of differing single-strand nucleic acid segments with a single-strand nucleic acid probe;
contacting the mixture with a membrane comprising at least one nanopore;
applying an electric field across the nanopore; and
measuring change in current through the nanopore during one or more nucleic acid translocation events.

2. The method of claim 1, wherein the translocation events comprise passage of an unamplified target single-strand nucleic acid segment hybridized with the single-strand nucleic acid probe.

3. The method of claim 2, wherein the translocation events comprise passage of a target single-strand nucleic acid segment hybridized with the single-strand nucleic acid probe in a 1:1 relationship.

4. The method of claim 2 further comprising quantifying the unamplified target single-strand nucleic acid segments of the mixture.

5. The method of claim 4, wherein quantifying comprises classifying a change in current below a predetermined threshold as translocation of the hybridized target single-strand nucleic acid segments and a change in current above the predetermined threshold as translocation of single-strand nucleic acid segments and recording the occurrence or frequency of each classified translocation event during the analysis.

6. The method of claim 4, wherein quantifying comprises classifying a nanopore dwell time below a predetermined threshold as translocation of the hybridized target single-strand nucleic acid segments and a nanopore dwell time above the predetermined threshold as translocation of single-strand nucleic acid segments and recording the occurrence or frequency of each classified translocation event during the analysis.

7. The method of claim 4, wherein quantifying is carried out in real time.

8. The method of claim 2, wherein the translocation events further comprise passage of self-hybridized single-strand nucleic acid demonstrating secondary structure.

9. The method of claim 1, wherein the at least one nanopore has a diameter between about 10 nm and about 20 nm.

10. The method of claim 1, wherein the mixture of single-strand nucleic acid segments is unpurified.

11. The method of claim 1, wherein single-strand nucleic acid probe comprises locked nucleic acid.

12. The method of claim 2, wherein the unamplified target single-strand nucleic acid segment comprises 50 to 500 nucleotides.

13. The method of claim 12, wherein the unamplified target single-strand nucleic acid segment is from a unique sequence of genomic or mitochondrial DNA from a pathogen species.

14. The method of claim 13, wherein the pathogen is a bacterium.

15. The method of claim 14, wherein the bacterium is Escherichia coli or Yersinia pestis.

16. The method of claim 13, wherein the unamplified target single-strand nucleic acid segment is genomic DNA of a prokaryotic or eukaryotic pathogen or mitochondrial DNA of a eukaryotic pathogen.

17. The method of claim 13, wherein the mixture of differing single-strand nucleic acid segments is derived from a water sample taken from a body of water.

18. The method of claim 13, wherein the membrane is formed of an inorganic material.

19. The method of claim 18, wherein the inorganic material is a ceramic.

20. The method of claim 1, wherein the membrane has a thickness of 1 nm to 100 nm.

21. The method of claim 1, wherein the membrane has a thickness of 100 μm to 500 μm.


SEQUENCE LISTING (35 sequences)

SEQ ID NO: 1 - DNA, 50 bp, Artificial Sequence
  Designed nucleic acid based on Escherichia coli.
agctaataccgcataacgtcgcaagaccaaagagggggaccttcgggcct

SEQ ID NO: 2 - DNA, 50 bp, Artificial Sequence
  Designed nucleic acid based on Escherichia coli.
aagaccaaagagggggaccttcgggcctcttgccatcggatgtgcccaga

SEQ ID NO: 3 - DNA, 50 bp, Artificial Sequence
  Designed nucleic acid based on Yersinia pestis.
aagagcaaagtgggggaccttagggcctcacgccatcggatgaacccaga

SEQ ID NO: 4 - DNA, 50 bp, Artificial Sequence
  Designed nucleic acid based on Yersinia pestis.
tctgggttcatccgatggcgtgaggccctaaggtcccccactttgctctt

SEQ ID NO: 5 - DNA, 60 bp, Artificial Sequence
  Designed nucleic acid based on 16S OTU 77+80.8.
tcaccagttttaccctaggcggctccttacggttaccgactttaggtacacccggcttcc

SEQ ID NO: 6 - DNA, 60 bp, Artificial Sequence
  Designed nucleic acid based on 16S OTU 80.8_20.
tcggccacaccgtggcaagcgccccccttgcggttaagctacctgcttctggtgcaacaa

SEQ ID NO: 7 - DNA, 60 bp, Artificial Sequence
  Designed nucleic acid based on 16S OTU 80.8_26.
ctagttaccagttttaccctaggcagctccttgcggtcaccgacttcaggcacccccagc

SEQ ID NO: 8 - DNA, 60 bp, Artificial Sequence
  Designed nucleic acid based on 16S OTU 80.8_2.
gaaccctgccgtggtaatcgccctccttgcggttaggctaactacttctggcagaacccg

SEQ ID NO: 9 - DNA, 60 bp, Artificial Sequence
  Designed nucleic acid based on 16S OTU 80.8_4a.
accagccttaccttaggacgctgcccccttgcggttggcgtgcatacttcgggtgcgacc

SEQ ID NO: 10 - DNA, 60 bp, Artificial Sequence
  Designed nucleic acid based on 16S OTU 80.8_4b.
accagccttaccttaggacgctgcccccttgcggttggcgcgcatacttcgggtgcgacc

SEQ ID NO: 11 - DNA, 60 bp, Artificial Sequence
  Designed nucleic acid based on 18S OTU 80.8_2.
caactctcgcggggagggatgtatttattagataaaaaaccaatgcgggttctgctcgcc

SEQ ID NO: 12 - DNA, 60 bp, Artificial Sequence
  Designed nucleic acid based on 18S OTU 80.8_20.
actttacgaaggggcgcttttattagatcaaaatcaatcaggagcaatcctgtttttgtg

SEQ ID NO: 13 - DNA, 60 bp, Artificial Sequence
  Designed nucleic acid based on 18S OTU 77_54.
gacccgacgcaaggacggtcgcatttattagaacaaagccatccggtccccgggaccgta

SEQ ID NO: 14 - DNA, 60 bp, Artificial Sequence
  Designed nucleic acid based on 18S OTU 80.8_34.
tgcgggacgagcgcatttattagaacaaaaccatccggactctcgcgagtccgttgctgg

SEQ ID NO: 15 - DNA, 60 bp, Artificial Sequence
  Designed nucleic acid based on 18S OTU 77_6.
cattttgggaaactatggctaatacatgcttacagaccttcgggttgtatttattagttt

SEQ ID NO: 16 - DNA, 60 bp, Artificial Sequence
  Designed nucleic acid based on 18S OTU 77_73.
gaccttcggaaagagcgcatttattagaccaaaaccagtcgagtttcggcttgtttgttg

SEQ ID NO: 17 - DNA, 60 bp, Artificial Sequence
  Designed nucleic acid based on 18S OTU GL_59.
caatacccttctggggtagtatttattagaaagaaaccaaccccttcggggtgatgtggt

SEQ ID NO: 18 - DNA, 60 bp, Artificial Sequence
  Designed nucleic acid based on 18S OTU GL_8.
acgaacgagcgcatttattagagcaaaaccaatcaggtttcggcctgtcttttggtgaat

SEQ ID NO: 19 - DNA, 60 bp, Artificial Sequence
  Designed nucleic acid based on 18S OTU 80.8_66a.
ccccgacttcggaaggggtgtatttattagataaaaaaccaatgcccttcggggctactt

SEQ ID NO: 20 - DNA, 60 bp, Artificial Sequence
  Designed nucleic acid based on 18S OTU 80.8_66b.
ccccaacttcgggaggggtgtatttattagataaaaaaccaacgcccttcggggcttctt

SEQ ID NO: 21 - DNA, 50 bp, Artificial Sequence
  Designed nucleic acid based on Cryposporidium parvum.
gatttctcataaggtgctgaaggagtaaggaacaacctccaatctctagt

SEQ ID NO: 22 - DNA, 50 bp, Artificial Sequence
  Designed nucleic acid based on Entamoeba histolytica.
gaaatgtcttattgacatcccctcagcattgtcccatgcttgaatattca

SEQ ID NO: 23 - DNA, 50 bp, Artificial Sequence
  Designed nucleic acid based on Giardia intestinalis.
cccacgcggcgggtccaacgggcctgcctggagcgctcccgtttcctcgt

SEQ ID NO: 24 - DNA, 50 bp, Artificial Sequence
  Designed nucleic acid based on Microsporidium sp. STF.
ctttatcatcggactcgcccctggccagcgctttcgcctctgtcgctcct

SEQ ID NO: 25 - DNA, 50 bp, Artificial Sequence
  Designed nucleic acid based on OUT LT1A42, multi-copy, identified as Cryptomonas sp., strain M420.
caagcagaaaggcacgcgcgcaccgtccaaccagaggctgacagttcaca

SEQ ID NO: 26 - DNA, 50 bp, Artificial Sequence
  Designed nucleic acid based on OUT LT1A4, multi-copy, identified as Cryptomonas ovata, strain CCAP 979/61.
gcacgcgcatgccgtccgaccagaggccgacagcccacacgcgcccaaaa

SEQ ID NO: 27 - DNA, 51 bp, Artificial Sequence
  Designed nucleic acid based on Bacillus anthracis strain S51.
gtgacagccgaagccgcctttcaattttcgaaccatgcggttcaaaatgtt

SEQ ID NO: 28 - DNA, 50 bp, Artificial Sequence
  Designed nucleic acid based on Francisella tularensis strain 3523.
aggctcatccatctgcgacacgccgaaagccacctttaatccacagatat

SEQ ID NO: 29 - DNA, 50 bp, Artificial Sequence
  Designed nucleic acid based on Legionella pneumophilia serogroup 6.
aatccttaaaagtcggtcgtagtccggattggagtctgcaactcgactcc

SEQ ID NO: 30 - DNA, 50 bp, Artificial Sequence
  Designed nucleic acid based on Salmonella typhimurium.
cttggtgagccgttacctcaccaacaagctaatcccatctgggcacatct

SEQ ID NO: 31 - DNA, 50 bp, Artificial Sequence
  Designed nucleic acid based on Vibrio cholerae CECT 514 T.
atcccacctgggcatatccggtagcgcaaggcccgaaggtcccctgcttt

SEQ ID NO: 32 - DNA, 50 bp, Artificial Sequence
  Designed nucleic acid based on Uncultured human fecal bacterium HF74.
tattcataaggtacatacaaaacaccacacgtggcgaactttattccctt

SEQ ID NO: 33 - DNA, 50 bp, Artificial Sequence
  Designed nucleic acid based on Uncultured human fecal bacterium HF8.
tattcataaagtacatgcaaacgggtatgcatacccgactttattccttt

SEQ ID NO: 34 - DNA, 50 bp, Artificial Sequence
  Designed nucleic acid based on Uncultured human fecal bacterium HF10.
tattcatacggtacatacaaaaaggcacacgtgcctcactttattcccgt

SEQ ID NO: 35 - DNA, 50 bp, Artificial Sequence
  Designed nucleic acid based on Burkolderia cepacia.
aggcccgaaggtcccccgctttcatccgtagatcgtatgcggtattaatc